(19) United States
(12) Patent Application Publication
(10) Pub. No.: US 2019/0027232 A1
(43) Pub. Date: Jan. 24, 2019

(54) SYSTEM AND METHOD FOR PROCESSING ELECTRONIC MEDICAL AND GENETIC/GENOMIC INFORMATION USING MACHINE LEARNING AND OTHER ADVANCED ANALYTICS TECHNIQUES

(71) Applicant: Celmatix Inc., New York, NY (US)

(72) Inventors: Piraye Yurttas Beim, New York, NY (US); Mark Adams, Norwalk, CT (US); Caterina Clementi, New York, NY (US)

(21) Appl. No.: 15/926,601

(22) Filed: Mar. 20, 2018

Related U.S. Application Data
(60) Provisional application No. 62/611,233, filed on Dec. 28, 2017.
(60) Provisional application No. 62/473,883, filed on Mar. 20, 2017.

(51) Int. Cl. G16H 10/40; G06F 19/22; G06F 19/24; G16H 10/60; G16H 15/00; G16H 50/30; G16H 50/20; G06F 19/28; G16H 80/00

(52) U.S. Cl. CPC G16H 10/40; G06F 19/22; G06F 19/24; G16H 10/60; G16H 80/00; G16H 50/30; G16H 50/20; G06F 19/28; G16H 15/00

(57) ABSTRACT

Embodiments of the present disclosure relate to a machine-learning system for processing medical information. The system comprises a communications interface configured to access electronic medical data. An automated retrieval processor is configured to analyze the electronic medical data to identify and retrieve relevant electronic data based on predefined search criteria. A learning processor is configured to update and optimize the automated retrieval processor based on received electronic metadata associated with the identified relevant electronic data. Other embodiments relate to a machine-learning method for processing electronic medical information. The method comprises accessing electronic medical data from a public database and/or a private database. In addition, the method comprises analyzing the electronic medical data to identify and retrieve relevant electronic data based on predefined search criteria. Also, the method includes performing adaptive learning based on received electronic metadata associated with the identified relevant electronic data.

### RELATED APPLICATIONS

This application is claims the benefit of and priority to U.S. Provisional Application No. 62/611,233, filed on Dec. 28, 2017, and U.S. Provisional Application No. 62/473,883, filed on Mar. 20, 2017, each of which is incorporated herein by reference in its entirety.

### BACKGROUND

Over the last few decades, the World Wide Web has become an important source of information within the medical community. Physicians primarily access the Internet at the point of care for medical records updating and to communicate with colleagues. The Internet gives medical professionals access to a vast amount of high-quality medical information, which could potentially aid medical decision making and patient care. It has been found that 51% of physicians claim that the Web has influenced treatment and assisted them in diagnostic procedures. The benefit and usability of medical information provided on the Internet increasingly relies on adequate content, quality evaluation, and the skilled selection of relevant websites and content.

However, physicians often fail to find the required information online. For example, barriers to medical information retrieval include inaccessibility of relevant information, questionable trustworthiness, and information overload. In addition, physicians either lack the time (general practitioner) or the skill (physician in training) to perform adequate data selection and evaluation when confronted with vast amounts of information. Moreover, because electronic medical data are generally non-numerical expressions of concepts and because there is no uniform method of expressing those concepts, search engines have traditionally lacked the capacity to recognize when a given electronic medical document satisfies a query. Furthermore, such databases and queries are further complicated by the growing amount of genetic and genomic data available to medical professionals. As such, current attempts at organizing, storing, and searching are not scalable with respect to the increasing amounts of available information. Consequently, the utility of such databases and queries diminish as a function data growth.

As is known, it is increasingly possible to provide better and more effective treatment to individuals by considering their genetic makeup as part of the totality of medical information considered by care providers. However, there are no known systems that integrate genetic/genomic data with clinical information that can be easily queried.

### SUMMARY

The present disclosure cures the aforementioned deficiency and provides systems for collecting, analyzing, storing, and providing an intersection of genetic and medical outcomes data to ensure that the information is both meaningful and available to those who need it. By providing systems that can effectively ingest and integrate these data types, the gap between cutting-edge biomedical research and care can be effectively bridged. In particular, the present disclosure enables clinician's decisions regarding current patient care to leverage real-time and ongoing cutting-edge biomedical research.

For example, aspects of the present disclosure relate to advanced analytics (such as machine-learning) tools, systems and methods for processing electronic medical information. In particular, embodiments of the present disclosure enable searches of vast amounts of medical related data in a manner that both harmonizes disparate terminologies and provides an adaptive learning search processor. The disclosed search processor is configured to update and optimize its search logic in response to receiving electronic metadata associated with results sets of previous searches. Advantageously, embodiments of the present disclosure provide a self-learning search processor that is capable performing adaptive learning to optimize future searching. Accordingly, the disclosed system provides increasingly accurate and valuable search results that allow physicians to provide the most up-to-date medical diagnoses and treatment plans.

In one embodiment, a machine-learning system for processing medical information comprises a communications interface configured to access electronic medical data. The system also comprises an automated retrieval processor configured to analyze the electronic medical data. The automated retrieval processor is further configured to identify and retrieve relevant electronic data based on predefined search criteria. The system also comprises a learning processor configured to update and optimize the automated retrieval processor based on received electronic metadata associated with the identified relevant electronic data.

In an aspect, the communications interface can be configured to access the electronic medical data from a public database, electronic medical records systems, a private database, or any combination thereof. The communications interface can further be configured to access a real-time medical data feed.

In other aspects, the system comprises a metadata tool configured to add electronic metadata to the identified relevant electronic data. The electronic metadata can comprise electronic identifiers corresponding to at least one of: a false-positive marking, a false-negative marking, at least one clinical data element, or any combination thereof. The at least one clinical data element can correspond to a predefined electronic annotation stored in a clinical data element store.

In additional aspects, the system can further comprise a phenotype/outcome data store configured to store and organize the identified relevant electronic data based on the added electronic metadata.

In further aspects, the system can further comprise a genome data store, and forked loader, and at least one set of parallelized parsers. The genome data store can be configured to store and organize genomic data. The forked loader can be configured to parse arbitrary file types into a predetermined format for loading genomic data into the genome data store. Each of the at least one set of parallelized parsers can be configured to parse a particular file type based on a parsing library corresponding to the particular file type.

Also, the system can comprise a query interface tool configured to access and retrieve information from at least one of: the phenotype/outcome data store, the genome data store, or any combination thereof.

Another embodiment of the present disclosure relates to a machine-learning method for processing electronic medical information. The method comprises accessing electronic medical data from a public database, a private database, or any combination thereof. The method also comprises analyzing the electronic medical data to identify and retrieve relevant electronic data based on predefined search criteria. Additionally, the method comprises performing adaptive learning based on received electronic metadata associated with the identified relevant electronic data.

In an aspect, accessing electronic medical data includes accessing a real-time medical data feed. Also, the received electronic metadata can be received from a metadata tool enabling addition of electronic metadata to the identified relevant electronic data.

In other aspects, the electronic metadata comprises electronic identifiers corresponding to at least one of: a false-positive marking, a false-negative marking, at least one clinical data element, or any combination thereof. In further aspects, the at least one clinical data element corresponds to a predefined electronic annotation stored in a clinical data element store. Also, the medical data can be at least one of: an electronic structured document and/or an electronic unstructured document.

In additional aspects, the method comprises storing and organizing the identified electronic relevant data based on the added electronic metadata in a phenotype/outcome data store. Other aspects of the method comprise storing and organizing genomic data in a genome data store, and parsing arbitrary file types into a predetermined format for loading genomic data into the genome data store. Parsing the arbitrary file types can include performing parallel parsing using at least one set of parallelized parsers, wherein each of the at least one set of parallelized parsers is configured to parse a particular file type based on a parsing library corresponding to the particular file type.

Also, the method can comprise enabling a query, via a query interface tool, to access and retrieve information from at least one of: the phenotype/outcome data store, the genome data store, or any combination thereof.

### BRIEF DESCRIPTION OF THE DRAWINGS

The foregoing will be apparent from the following more particular description of example embodiments of the disclosure, as illustrated in the accompanying drawings in which like reference characters refer to the same parts throughout the different views. The drawings are not necessarily to scale, emphasis instead being placed upon illustrating embodiments of the present disclosure.

FIG. 1 illustrates an example environment in which a machine learning system operates to facilitate search and retrieval of medical related information in accordance with an example embodiment of the present disclosure.

FIG. 2 illustrates a machine-learning method for processing electronic medical information in accordance with an example embodiment of the present disclosure.

FIG. 3 is a logical block diagram of an electronic medical document according to an example embodiment of the present disclosure.

FIGS. 4A-C are architectural diagrams of example systems for providing personalized medicine in accordance with an example embodiment of the present disclosure.

FIG. 5 illustrates a logical block diagram of a process for loading electronic medical data into a genome database 435.

FIGS. 6A-B illustrate an example data structure for storing and accessing medical related information in a data store or memory according with an example embodiment of the present disclosure.

FIG. 7 illustrates a detailed block diagram of electrical systems of an example computing device in accordance with an example embodiment of the present disclosure.

FIG. 8 is a flow diagram of a method for comprehensive literature review and meta-analysis implementing an adaptive biocuration technology in accordance with an example embodiment of the present disclosure.

FIG. 9 is a chart that defines distinct levels of clinical association evidence in accordance with an example embodiment of the present disclosure.

FIG. 10 is a flow diagram of an example method for conducting a statistical validation analysis.

FIG. 11 is a chart that shows results from a search performed using natural language processing techniques in accordance with an example embodiment of the present disclosure.

### DETAILED DESCRIPTION

A description of example embodiments of the present disclosure follows.

The Internet continues to grow, driven by ever greater amounts of online information and knowledge, commerce, entertainment and social networking. With respect to the medical field, the Internet has enabled easy access to medical research and information. For example, the medical field has benefited from the sharing of clinical research and medical records information to assist with diagnosing and treating patients for a variety of medical issues. However, the Internet has also provided some unintended consequences. In particular, although the Internet has allowed physicians to access numerous amounts of data (e.g., millions of journal articles, clinical trial data, and/or medical records); unfortunately, the amount of data available for review is so vast that it is impractical for medical practitioners to efficiently search through the extensive amount of data to find information relevant to a specific patient's needs.

Embodiments of the present disclosure enable searches of vast amounts of medical related data to return real-time results that enable a physician to provide up-to-date health care services. Specifically, embodiments of the present disclosure relate to a searching engine that utilizes a machine-learning system to adaptively optimize its searching logic. Accordingly, the disclosed system provides increasingly relevant and accurate search results that allow physicians to provide the most up-to-date medical diagnoses.

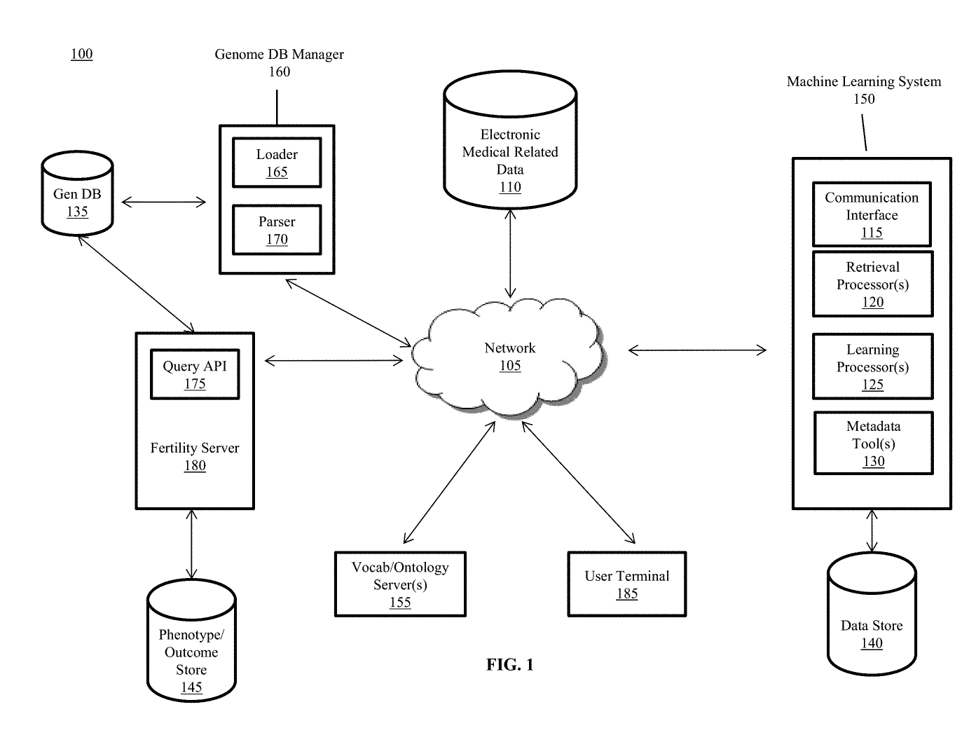

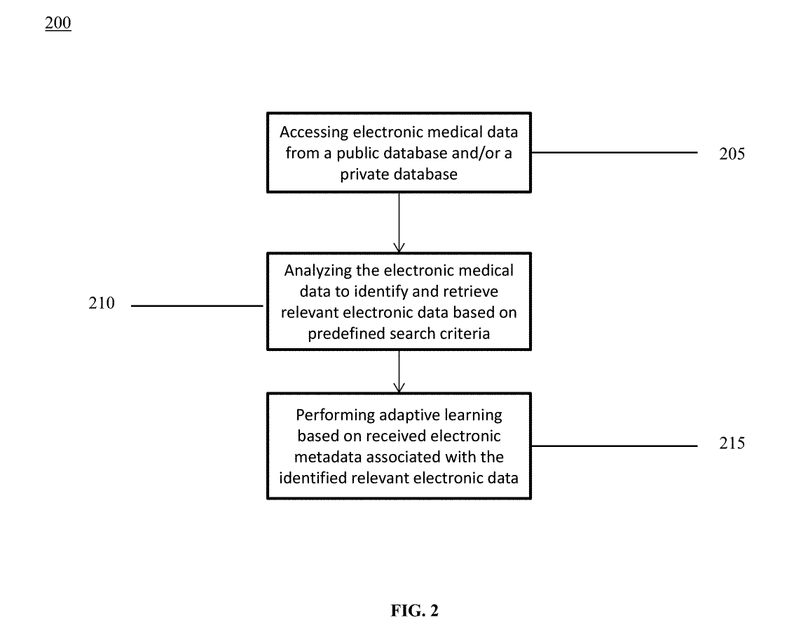

FIG. 1 illustrates an example environment 100 in which a machine learning system 150 operates to facilitate search and retrieval of electronic medical data (e.g., genomic data, patient medical records, physician/nurse notes, phenotype/outcome data, clinical research, and medical publications).

The machine learning system 150 comprises a communication interface, retrieval processor(s) 120, learning processor(s), and metadata tool(s) 130. The machine learning system 150 is communicatively coupled to a network 105. The network 105 can be any computing network such as a wide area network (e.g., the internet), a local area network (LAN), or any combination thereof.

The communication interface 115 is configured to enable the machine learning system 150, via the network 105, to access public and/or privately available electronic medical related data. For instance, the public and/or privately available data can be retrieved from an electronic medical related data store 110. The medical related data store 110 can be a single database or a collection of various databases having information stored therein from disparate sources. In an example, the medical related data store 110 can be a publicly accessible database such as PubMed® that stores a variety of electronic medical documents (e.g., journal articles, clinical research data, medical periodicals, etc.). In addition, the medical record data store 110 can correspond to private electronic medical record databases, electronic health records databases, clinical trial management systems databases, or any combination thereof. In some examples, the medical record data store 110 can stream real-time physician messaging (e.g., Health Level 7 (“HL7”) messages).

The machine learning system 150 includes retrieval processor(s) 120 configured to perform searches over the network 105 to obtain, for example, electronic information, medical records, and/or research related to a particular medical field such as fertility. The retrieval processor(s) 120 can be configured to perform a natural language search to search for and retrieve relevant electronic information, medical records, and/or research. In response to performing the natural language search, the retrieval processor(s) 120 retrieves electronic medical data. The electronic medical data can be electronic documents having a structured format, unstructured format, semi-structured, or a format of any combination thereof.

Due to the vast amount of data available over the network 105, some of the results can be irrelevant. Accordingly, such data can be electronically encoded, via a metadata tool 130, as a false positive. Learning processor(s) 125 can then analyze the metadata to optimize and update searching engine logic of the retrieval processor(s) 120. For example, the metadata can include an index of terms associated with a particular electronic document marked as a false positive. The learning processor(s) 125 can identify a commonality in term usage/frequency between each electronic document marked as a false positive. The identified commonality can be encoded into the search engine logic of the retrieval processor(s) as a filter. Accordingly, any document that has a similar term usage/frequency can be discarded from a potential future result set.

For particular applications, such as searches related to reproductive health, additional culling of the results may be needed. In an example, terms/phrases may have more than one meaning or use. Thus, a natural language search that is based on keywords and/or phrases will retrieve all electronic documents including the particular keywords and/or phrases. Accordingly, the machine learning system 150 is provided with a data store 140 that includes a library of electronic clinical data elements to facilitate the additional culling. The library of electronic clinical data elements includes semantics of terms/phrases unique to a particular medical field (e.g., reproductive health) which is the subject of a current search. The metadata tool(s) 130 enable a reviewer to electronically encode the electronic documents with metadata corresponding to the electronic data elements. For instance, the clinical data elements can be electronic annotations that are used to categorize an electronic document based on predefined semantics that are relevant to the particular medical field. The learning processor(s) 125 is further configured to analyze the electronic metadata to provide an additional layer of adaptive learning to the retrieval processor(s) 120. Particularly, the metadata can be used to determine a particular usage of a term/phrase that is consistent with or inconsistent with the semantics of the clinical data elements. The determined usage can be used to process electronic documents in future searches to provide for an increasingly relevant result set of electronic medical data.

In one embodiment, the retrieval processor(s) 120 utilize natural language processing (NLP) to analyze published literature and assign a score of relevance to each publication. The retrieval processor(s) 120 returns results based on the score of relevance. For instance, the processor receives a selection of a topic related to a search to be performed using NLP. Additionally, the processor(s) receives or accesses keywords applicable to the search to determine the relevancy of any particular document (i.e., search result). The keyword can include one or both of positive terms and negative terms. Positive terms are those keywords that add to the relevancy of a particular document (i.e., increase the score of relevancy), while negative terms are those keywords that detract from the relevancy of a particular document (i.e., lower or do not add to the score of relevancy).

In one aspect, a corpus of published articles/documents (i.e., a training set od documents) associated with the selected topic is screened to identify the positive and negative keywords. The documents are divided into two groups: 1) papers relevant and 2) papers irrelevant to the selected topic. NLP is performed on each group to identify keywords that are enriched in each group. Accordingly, those keywords in the papers relevant group are positive keywords (i.e. relevant to the search topic), while those keywords in the irrelevant group are negative keywords (i.e., irrelevant to the search topic).

In one example, each group is analyzed to determine a frequency (i.e., number of occurrences) of terms. In each group, those terms that meet or exceed a certain frequency threshold are labeled as a keyword. Additionally, any known or yet to be known method to identify keywords in a group of documents can be used. NPL can further comprise evaluation of parameters related to the keywords such as the presence and position of each keyword in any section of document (e.g., the title, abstract, text, etc. of the document), total number of positive and negative keywords, number of unique positive and negative keywords, combinations of positive and negative keywords, etc. Additionally, a weight is given to each parameter based on how strongly each parameter associates with relevant or irrelevant documents. A strength of a parameters association with the relevancy or irrelevancy of a document can be based on historical data, a relative occurrence of each parameter in a set of documents, or any other known or yet to be known weighting method. Thus, the retrieval processor 120 uses the keywords, parameters, and weightings of each parameter to give each document a score of relevance.

Table 1 below is an example list of relevant and irrelevant keywords identified from a corpus of documents associated with the topic “ovarian biology and reproduction”. Those documents that discussed ovarian biology and reproduction were placed in the papers relevant group, while those documents that did not were placed in the papers irrelevant group. NLP was performed on each group, and the relevant keywords were obtained from the papers relevant group and the irrelevant keywords were obtained from the papers irrelevant group.

The list of Table 1 was further supplemented by an additional training set of documents that contained information on either genes that are known to play a role in ovarian biology (positive set, likely to be relevant) or on genes for which a role in ovarian biology was not known (negative set, likely to be not relevant). The presence of positive and negative keywords in each publication, total count of positive and negative keywords and the number of unique positive and negative keywords were evaluated for each publication.

TABLE 1
Keywords related to Ovarian Biology and Reproduction

| Relevant (positive) keywords | Irrelevant (negative) keywords |
|---|---|
| antral follicle | adenocarcinoma |
| cumulus cell | adenoma |
| cumulus oophorus | bladder |
| estradiol | breast cancer |
| estrogen | breast ovarian |
| fallopian tube | cancer |
| fertility | carcinogenesis |
| fertilization | carcinoma |
| follicle stimulating hormone | cardiac |
| follicular fluid | cardiomyopathy |
| gonadal | chemotherapy |
| gonadotropin | colorectal |
| granulosa cell | coronary |
| infertility | dermal |
| ivf | facial |
| menopause | follicular thyroid |
| oocyte | gastric |
| oocyte matur | hair follicle |
| oocyte quality | low grade |
| ovarian failure | lung |
| ovarian function | lymphoma |
| ovarian hyperstimulation | malignanc |
| ovarian insufficiency | malignant |
| ovarian reserve | melanoma |
| ovulation | mesothelial |
| ovulatory | metastasis |
| polycystic ovar | metastatic |
| pregnancy | neck |
| preovulatory | oral |
| primordial follicle | ovarian cancer |
| | ovarian carcinoma |
| | ovarian tumor |
| | papilla |
| | platinum |
| | prostate |
| | right coronary |
| | risk ovarian cancer |
| | scalp |
| | schizophrenia |
| | serous |
| | skin |
| | thyroid cancer |
| | thyroid carcinoma |
| | tumor cells |
| | tumor suppressor |

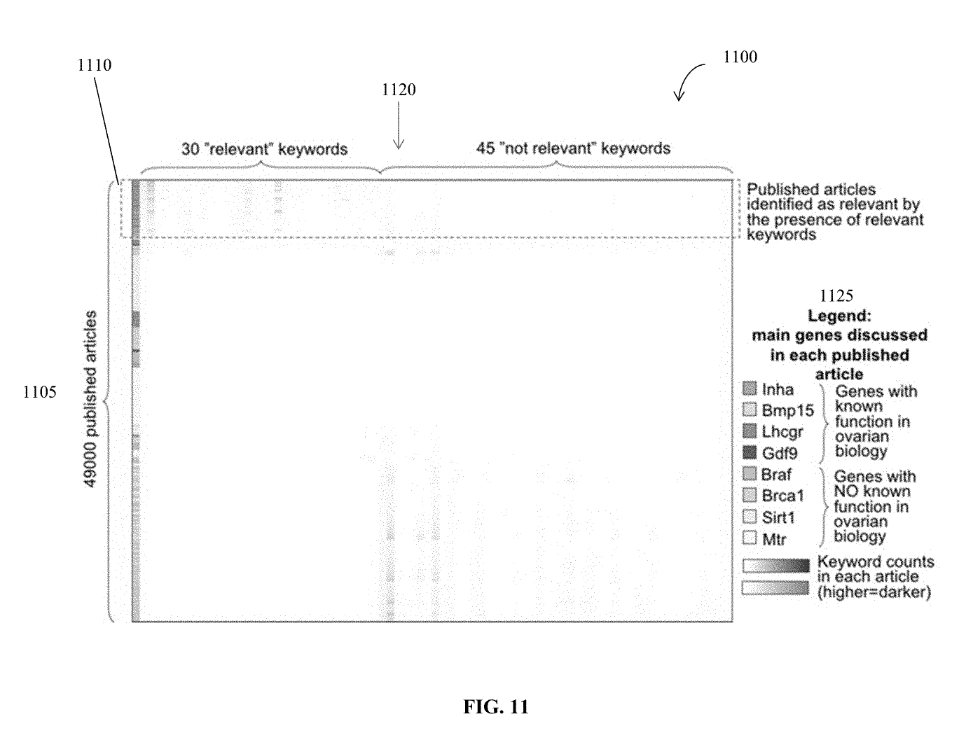

FIG. 11 is a chart 1100 showing a relationship of keyword relevancy for each article 1105.

The chart 1100 identifies a group of articles 1110 (bounded by the dotted rectangle) among which the positive set articles (rows 1110 with a darker shaded cell) shows a significant enrichment compares to the negative articles (rows 1110 with a lighter shaded cell). The identification is based on a relationship mapping 1125 between keywords 1120 (i.e., those listed in Table 1 below) and each article 1110.

A skilled artisan understands that the NLP disclosed herein is not limited to the biological examples described herein, and can be applied to any search for any topic in any field.

Additionally, the metadata corresponding to the clinical data elements can be used to harmonize electronic documents for storage in a phenotype/outcome data store 145. In this example, the phenotype/outcome data store 145 includes a collection of electronic data relevant to fertility. To that end, the phenotype/outcome data store includes a schema corresponding to the predefined semantics contained in the data store 140. The schema enables the phenotype/outcome data store 145 to parse the metadata of the electronic data retrieved by the retrieval processor(s) for logical storage according to the predefined semantics. Advantageously, the machine learning system 150 allows electronic medical documents having unknown semantic uses of terms/phrase to be harmonized/translated in the outcome store 145 for later search and retrieval by physicians.

The schema of the phenotype/outcome data store 145 can include a schema architecture that leverages a reactive streams model and can be built on a Confluent reference architecture. Advantageously, this architecture enables the automated processing and re-processing of the data being analyzed by the system, without the need to reconfigure the underlying systems. Furthermore, new processing steps can be flexibly added to accommodate transformation, encryption, and real-time analysis without disruption of the existing systems. By leveraging the “Schema Repository” provided by the platform, data being processed by the stream (e.g., the data being retrieved by the retrieval processor(s) 120) can be validated against the standardized common data elements, facilitating the analysis of data from a wide range of sources. Together, these capabilities provide rapid scalability, flexibility, and minimize the amount of software engineering needed to add new data sources and data types.

For example, the schema architecture uniquely leverages high performance systems used to support real-time social networking and e-commerce platforms (e.g., LinkedIn and Twitter) to enable high volumes of data (e.g., data generated from next generation DNA sequencing (NGS)) to be accommodated flexibly, simply, and in a scalable manner.

Specifically, the schema can utilize specialized data pipelines to receive streaming data. Each pipeline can be uniquely interfaced with a particular data source. such that the incoming data streams can be logically integrated into a data store. For instance, each interface can include logic specific to the data being received in a particular pipeline. In this way, the architecture can efficiently utilize computing resources (e.g., memory and CPU units) to implement logic used to process incoming data. Further, the data store can be a distributed storage system or single partitioned database that can efficiently organize, store, and correlate vast amounts of disparate data.

As illustrated, the environment 100 includes a user terminal 185 communicatively coupled to the network 105. In an example, the user terminal 185 can be that of a reproductive health clinician. The reproductive health clinician may have a patient/couple with a particular genomic and phenotypic profile. Accordingly, the clinician may wish to provide the patient/couple with, for example, the quantifiable and/or qualitative metrics of the likelihood of achieving live birth corresponding to a particular fertility treatment. To that end, the disclosed system ingests and integrates genetic and medical outcome data to provide a quantifiable likelihood of success of treatment that is specific to a certain patient. In contrasts, current naïve systems can only enabled clinicians to provide generalized metrics associated with a likelihood of success of a prescribed treatment.

In use, the reproductive health clinician issues a search query to a reproductive health server 180. The search query can include medical information of the patient/couple such as genetic data (including but not limited to genetic test results and DNA sequences), blood pressure, body mass index, etc. In response to receiving the search query, the reproductive health server, via a query API 175, performs a search of a genome data store 135 and the phenotype/outcome data store 145. The query API 175 returns a result set that includes information associated with results of fertility treatments that is correlated to the patient/couple genomic information and/or phenotype information. This correlation is based on a mapping of genomic data in the genome data store 135 and phenotype/outcome information in the phenotype/outcome store 145. In some examples, the system 150 can utilize advanced correlation techniques such as deep learning, or machine classifiers such as random forests, as well as statistical analysis tools (e.g., Principal Component Analysis). The mapping is based on the patient/couple genomic information and/or phenotype information.

In an example embodiment, the genome data store 135 is communicatively coupled to a genome database manager 165. The genome data store 135 stores and organizes genomic data from a variety of data sources (e.g., clinical partners, internal samples, etc.) that may need to be translated into a file format compatible with a schema of the genome data store 135. Accordingly, a loader 165 of the genome database manager 160 is configured to parse arbitrary file types and transform them into the format corresponding to the schema of the genome data store 135. In the current example, the loader 165 utilizes parser 170 to parse the arbitrary file types. The parser 170 can include a parsing library (not shown) that includes formatting logic corresponding to particular file types. Thus, the parser 170 identifies a particular file type of electronic genome data and reformats (i.e., parses) the electronic data into the format compatible with the schema of the genome data store 135.

In some embodiments, the database manager 165 can be communicatively coupled to the phenotype/outcome data store 145 and the data store 140. In such embodiments, the database manager 165 includes loader(s), parser(s) 179, and parsing libraries specific to process incoming data for storage in each data store.

As stated herein, the disclosed system enables processing and storage of real-time data streams. In some instances, data elements are added to a data stream asynchronously and potentially on a real-time basis. The system 150 is configured to transform these data elements (for example, by “stream workers” (not shown) which are processing tools that identify and remove any identifying elements (e.g., patient specific information) from medical data) and then create a new stream of data that is stripped of any patient identifying elements. This stream (and the data repository/database where that information is ultimately stored) can also include its own level of encryption and access controls consistent with stripped data. The new stream may now itself be processed by additional stream workers which use the semantics data stored in the vocab/ontology server(s) 155 to transform individual, unique data elements from a given source (in this example a specific electronic medical records system—which generally has its own unique representation of medical data) into a data representation formation of the present disclosure. Notably, the data representation format can be a shared/unified standard for representing medical data. A simple example could be transforming the records from one system which measures patient height in inches, to the system standard, which represents height in centimeters. The business rules that these transformations follow make use of the vocabulary/ontology repository 155, which also can be used to validate the data flowing in the data streams.

FIG. 2 illustrates a machine-learning method 200 for processing electronic medical information. The method 200, at 205, includes accessing medical data (e.g., via the machine learning system 150 of FIG. 1) from a public database and/or a private database (e.g., the Public/Private Database 110 of FIG. 1). At 210, the method 200 includes, analyzing the electronic medical data to identify and retrieve relevant electronic data based on predefined search criteria. In an example, the retrieval processor(s) 120 can be used to analyze the electronic medical data. The method 200 also includes performing adaptive learning based on received electronic metadata associated with the identified relevant electronic data. For example, the learning processor(s) 125 can automatically optimize searching logic of the retrieval processor(s) 120.

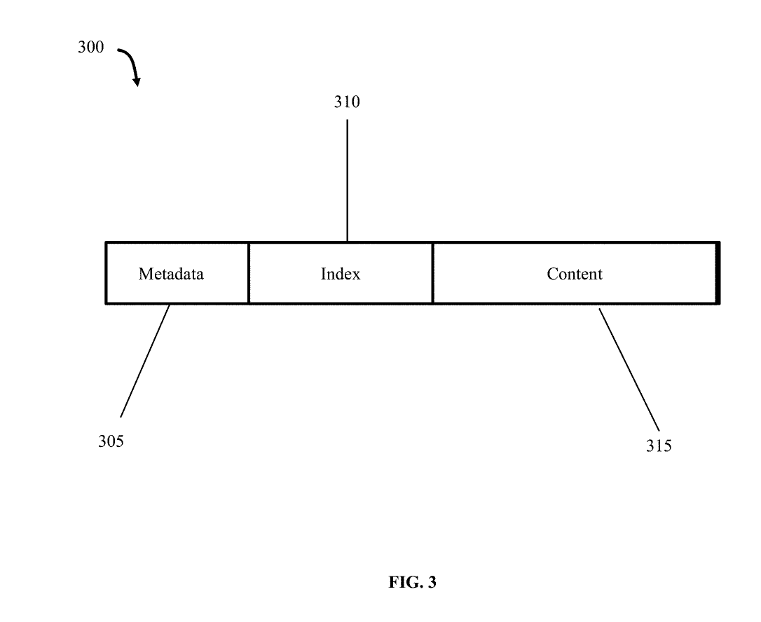

FIG. 3 illustrates an electronic medical document 300 that includes content 315, an index 310, and metadata 305. A medical records searching tool (e.g., the machine learning system 150 of FIG. 1) can perform searches for electronic medical data from public and/or private sources. In response to a search query, the retrieval processor 120 scans the content 315 of the electronic medical document 300 using, for example, natural language searching tools. The retrieval processor 120 can create an electronic index 310 that is encoded into or appended onto the electronic document 300. The index 310 includes electronic data mapping keywords and/or phrases identified in the document. The keywords and/or phrases can include those that correspond to the search query. However, as stated herein, search results can include data that is irrelevant to the search query being performed (e.g., due to non-standardized semantics of medical terminology). For instance, the content 315 of the electronic medical document may not be relevant to a particular search related to the reproductive health of a patient/couple.

Accordingly, embodiments of the present disclosure provide a metadata tool 130 that enables a reviewer to encode metadata 305 onto the document 300 that includes annotations to categorize the electronic document 300. The metadata 305 can be annotations based on a predefined semantics library stored in a data store (e.g., the data store 140 of FIG. 1). The metadata 305 and the index 310 can be used by the learning processor(s) 125 to identify an “electronic fingerprint” of terms/phrase usage within the document. Accordingly, if the electronic document 300 is determined to be irrelevant by a reviewer, the learning processor(s) can compare “electronic fingerprints” of other electronic documents (not shown) that are similarly marked as irrelevant. If a common usage of terms/phrases is identified in a threshold number of documents electronically marked as irrelevant, the learning processor(s) 125 can encode a logical filter associated with the common usage of terms/phrases into the retrieval processor(s) 120. The logical filter enables the retrieval processor(s) 120 to discard electronic documents having an electronic index 310 that matches parameters of the logical filter in a related future search query.

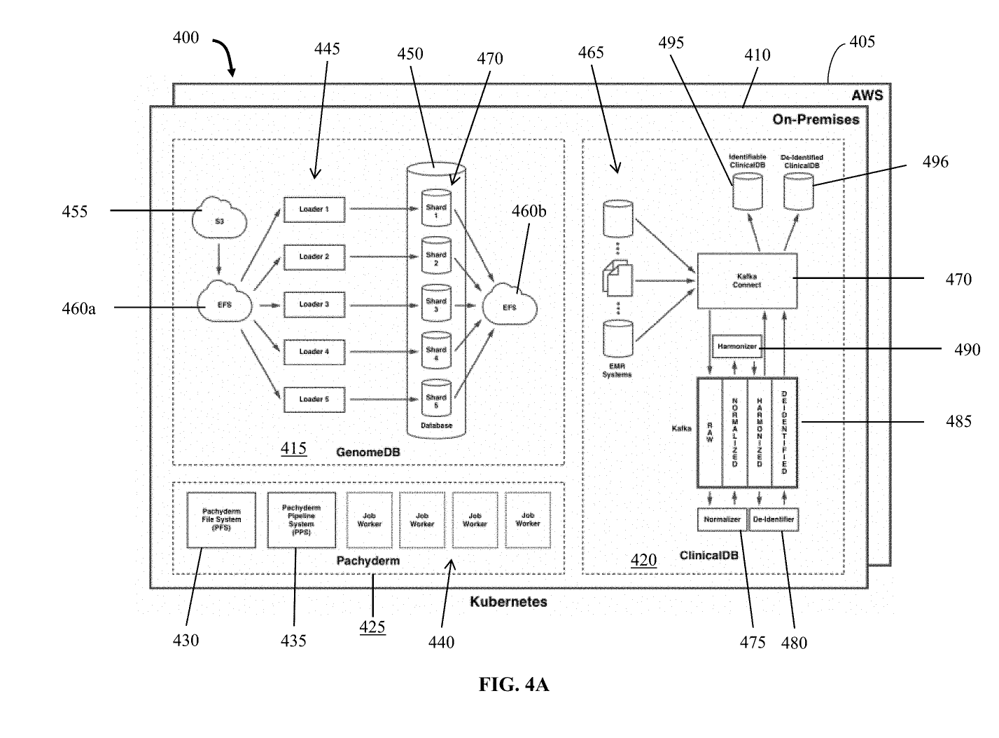

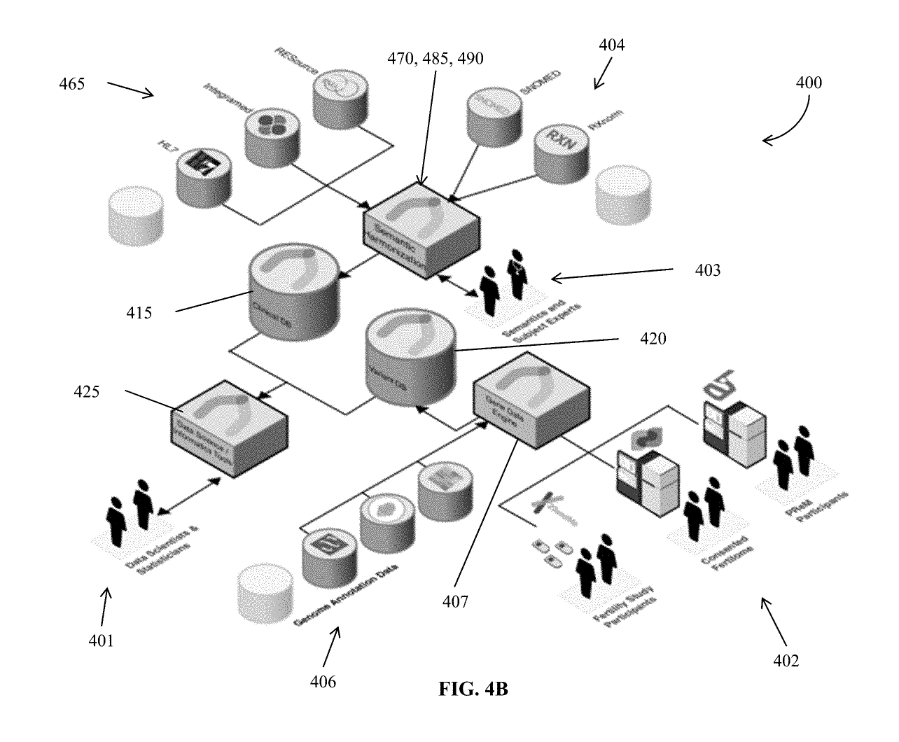

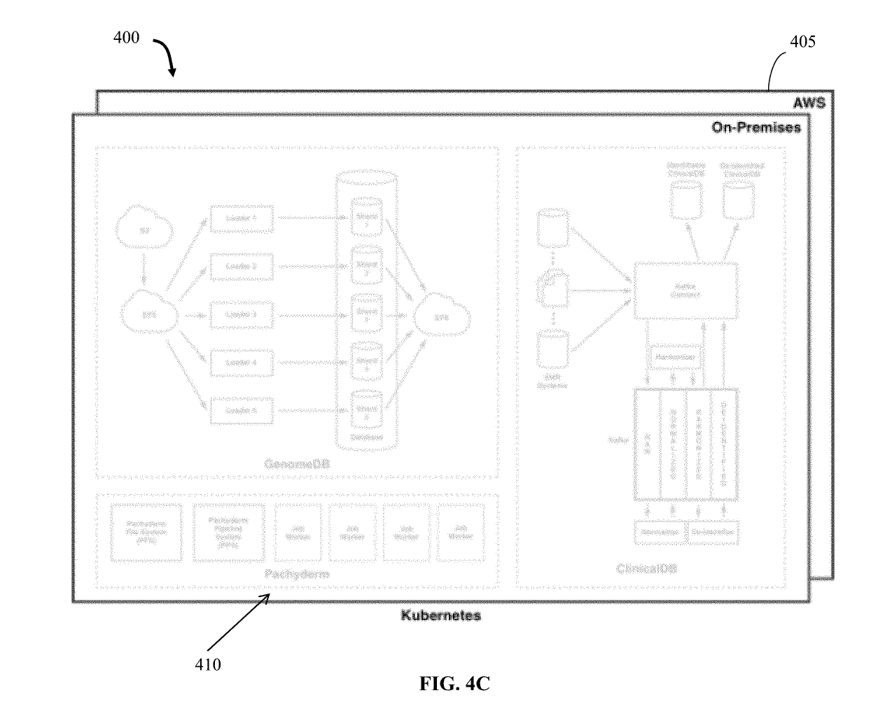

FIGS. 4A-C are architectural diagrams of an example system 400 for providing personalized medicine in accordance with an example embodiment of the present disclosure.

The system 400 is an integrated and query-able repository of genetic, clinical, biological, and literature annotation data, that is configured to access a community of contributors that include patients, clinical study participants, clinicians, and researchers. The system 400 enables collaboration between individuals within an entity and those individuals external to an entity (e.g., a research entity and a pharmaceutical company).

The integrated and query-able repository defined by the system 400 is thus a learning health system that is able to leverage real-time external information to benefit and supplement research being conducted within an organization to provide patients with personalized medical care. For example, the system integrates genetic variant data, clinical outcome data, biological annotations, and clinical annotations such that physicians providing care for patients, e.g., infertility patients can provide personalized medical care using the most relevant and up-to-date medical data possible, e.g., by leveraging genetic data and current clinical data.

Although the system 400 is described herein with respect to fertility, a skilled artisan understands that the system 400 can be applied to help provide personalized medical treatment across many disease areas such as oncology, cardiovascular, emergency medicine, and others. With respect to fertility, system 400 efficiently performs large-scale data mining using natural language machine learning searching tools described herein to retrieve data that can identify clinical relationships with, e.g., biomarker discovery such as those related to a fertility-centric biocuration. The clinical relationships can then be compared with a particular patient's electronic medical records (EMR) to determine outcome data for a particular fertility patient which is stored in, e.g., a reproductive knowledge database of system 400. The comparison of clinical relationships with the patient's EMR to determine the patient's outcome data is used to provide personalized medical care for that particular patient. Further, that patient's outcome data can then later be used to facilitate the development of personalized medical care for another patient.

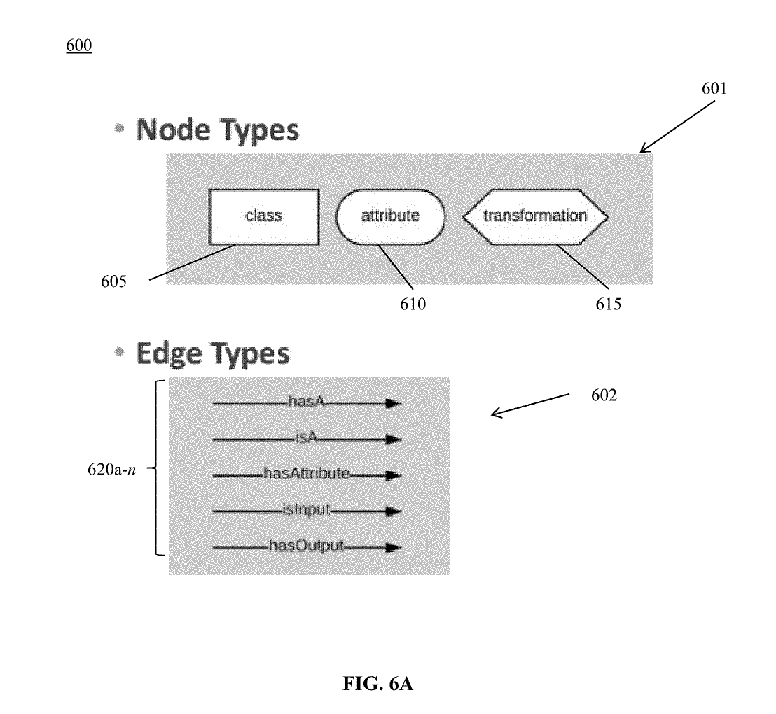

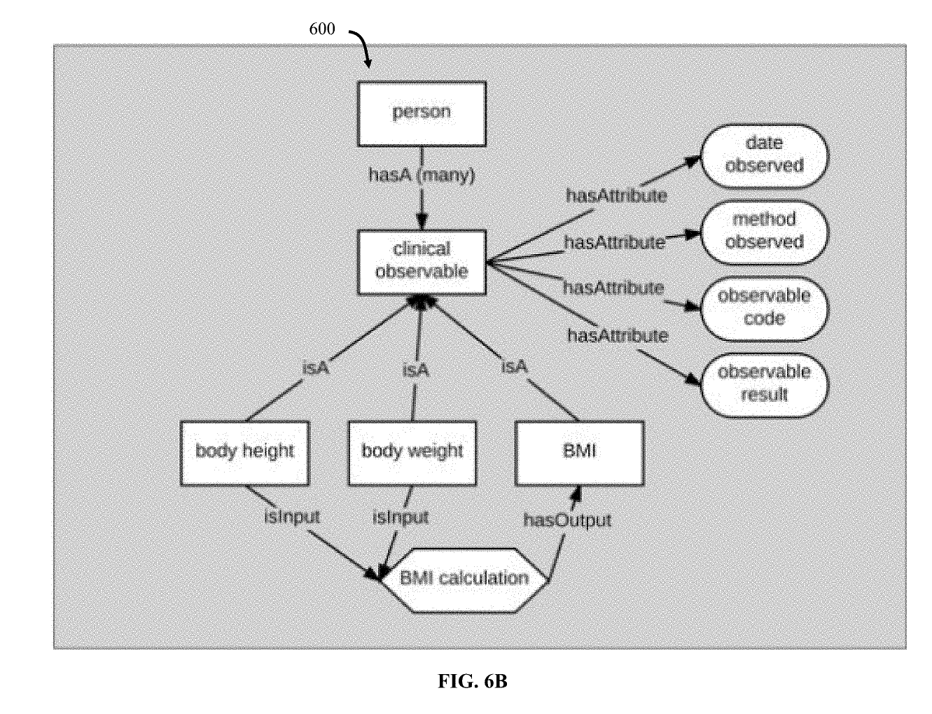

The system 400 performs such personalized care by efficiently searching, storing, and retrieving information using a unique data structure having a data model as described below in FIGS. 6A-B and a sharded repository defined by a genomic database repository (e.g., repository system 415 of FIG. 4A-B).

For example, referring to FIG. 4A-C, the system 400 comprises a local server system 410 that leverages resources, as needed, from a remote server system 405. The local server system 410 includes a genomic database repository system 415, a clinical database repository system 420, and a pachyderm interface system 425.

Referring to FIG. 4C, the local server system 410 includes a processing layer configured to manage applications, storage, resource quotas across an entire cluster of servers. The server system 410 comprises a plurality of containers provisioned within a single computing machine and is configured to interface with and access the remote server system 405 such that resources such as virtual machines (VMs) are made available as needed based on computing resource demands that are greater than that the local server system 410 can fulfill. In such circumstances, workload is split across private clouds (e.g., local hardware) and public clouds (e.g., remote VMs). Additionally, the remote server system 405 enables auto-scaling to scale/up computing resources such as memory and processing power as necessary.

The genomic database repository system 415 includes a cloud-based object storage 455 from which a file system 460a implementing, e.g., an elastic file system (EFS), obtains genetic data comprising genomic data, e.g., variant information, and reference sequences, which can be stored in documents having different formats, e.g., variant call format (VCF), FAST-ALL (FASTA) format, and any other format. The genetic data from the cloud-based object storage 455 can be obtained from a community of data providers 402 that include, e.g., fertility study participants, fertilome genetic tests, and personalized reproductive medicine (PReM) initiative participants.

The file system 460a uses loaders 445 to ingest the genetic data. Each of the loaders 445 can correspond to a respective shard 470 of a genome data store 450. Each of the shards 470 partitions the data store (e.g., database) 450 such that each shard ingests and stores a chromosome or set of chromosomes. Specifically, each shard can contain any chromosome. For example, the first shard ingests and contains chromosomes 1, 6, 11, 16, and 21, and the other shards ingest and contain other chromosomes. In other examples, each of the shards 1-5 of the shards 470 ingest and store one of chromosomes 1, 6, 11, 16, and 21. The loaders 445 include core resources such that loading times are scaled proportionally with a number of genetic data samples/documents divided by a number of available cores. The file system 460a is configured to scale up/down computing resources based on a load on the file system 460a based on a number of documents being loaded into the data store 450. Each of the loaders 445 can process the genomic data in parallel without cross-shard interference, e.g., such that each of the loaders 445 can process a single genomic document or a set of genomic documents in parallel to ingest its respective chromosome information. Using the five shard architecture depicted by FIG. 4A, the system 400 can load approximately two-hundred thousand documents per second per shard or about one-million documents per second overall.

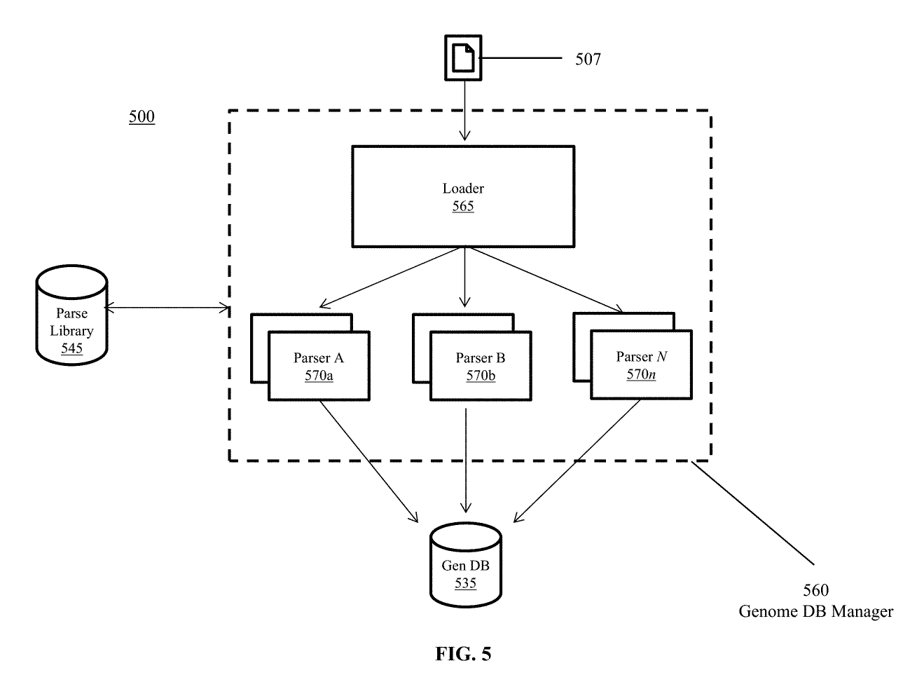

FIG. 5 illustrates a logical block diagram of a process 500 for loading electronic medical data 507 into a genome database 535. As illustrated, a genome database manager 560 is communicatively coupled to the genome database 535. The genome database manager 560 is configured to process the electronic medical data 507 for storage in the genome database 535. The genome database manager includes loader circuitry/logic 565 and sets of parallelized parsers 570a-n that are configured to process the electronic medical data 507 for loading into the genome database 535.

In an example, the electronic medical data 507 can be genomic data, genome reference sequences, or any combination thereof. The genomic data can be in a VCF 4.2 file format. The reference sequences can be in a FASTA format. The sharding structure of the genomic database 535 is defined by a certain number of chromosomes per shard, based on the overall size of the genomic data contained in each chromosome. This allows for a uniform distribution of the genomic data across any given number of shards. In some embodiments, the implementation of software-defined storage (SDS) architecture allows for horizontal scalability by scaling 1000s of exabytes of genomic data storage independent of the underlying hardware.

Advantageously, such sharding facilitates analysis of the data by retaining specific chromosomes for local processing, while allowing for horizontal scalability with a chromosome to machine ratio of up to 1:1. In some embodiments, the genomic database 535 can shard by centromere as well as chromosome. Such sharding allows for a chromosome to machine ratio of up to 0.5:1.

A portion of the genome database 535 in which genomic data is loaded can be a MongoDB type database that is configured to receive documents with a schema similar to the VCF 4.2 file format. In an example, the loader 535 includes forked parsers 570a-n to allow for parsing of arbitrary file types into, for example, a BSON document that can be directly inserted into the genome database 535. To that end, each set of the parallelized parsers 570a-n are associated with a particular file type. In particular, each set of the parallelized parsers 570a-n receive parsing logic for a distinct file type from the parse library 545 that is communicatively coupled to the genome database manager 560.

Advantageously, the present disclosure enables fast and efficient processing by utilizing a database structure that enables parallel processing (e.g., a MongoDB). For example, the database structure is “sharded” into multiple parallel systems. Each shard of the database is provided with a parser by the loader 535 specific to the machine instruction architecture of each shard. For instance, each parser is includes logic using assembly language specific to each shard. Thus, each parser has a 1:1 correspondence between itself the architecture of the shard's machine code instructions. Furthermore, since the large volume of genetic information is structured in such a way that its parsing and storage can be easily spread over multiple systems (i.e. the records are very simple and repetitive, and also have few dependencies between one record—genetic variant—and another) the processing framework benefits from a parallel processing system, and can be carried out quickly by leveraging many small computer systems at once (i.e., each shard can be implemented as a small computer system).

Referring back to FIGS. 4A-C, the file system 460a dynamically expands its storage space as an amount of data being stored increases. Accordingly, the file system 460a monitors data storage requirements and anticipates a need to increase its available free storage space such that the file system 460a does not run out of space. For example, the file system 460a can automatically acquire additional storage, e.g., cloud-based or local storage, when available/free storage space falls below a pre-determined threshold. The pre-determined threshold can be a percent available/free storage space with respect to total storage space. The file system 460a can also determine either a rate or change in a rate at which free storage space is being consumed such that the pre-determined threshold is adjusted to ensure that additional storage space can be acquired and provisioned prior to reaching a current storage space limit of the file system 460a.

Once chromosome information has been ingested, the file system 460a is configured to support queries across each of the fields present in the distinct genomic document formats such that genotype output is returned in seconds, and both genotype and reference sequence information is output in minutes. For instance, the file system 460a takes advantage of data parallelism to split up computation between nodes, e.g., shards 1-5 such that queries are distributed between shards, and output from each shard is combined to serve results.

The clinical database repository system 420 has a reactive streams architecture that comprises clinical databases 495-496, stream processing platform 485, stream connector 470, external data stream sources 465, and processing units comprising a data normalizer 475, harmonizer 490, and de-identifier 485. The reactive streams architecture enables the system 420 to receive real-time streams of data from the external data stream sources 465 via the stream connector 470 such that the data can be transformed and deposited in a data “sick” or persistent data store, as defined by databases 495-496, for later query and analysis. For example, the clinical databases 495-96 store and allow access to both harmonized data elements, and raw data from clinics to facilitate exploratory analysis.

The clinical databases are comprised of an identifiable clinical database 495 and de-identified clinical database 496 that contain raw and harmonized clinical data of patients received from the external data stream sources 465. Specifically, the clinical databases 495-496 provide a single source of clinical data elements in both patient identifiable and de-identifiable forms (i.e., clinical data stripped of all patient identifiable information such as name and social security number). The clinical databases 495-96 are updated in real-time through change data captured in, e.g., EMR systems that are included with the external data stream sources 465.

The stream processing platform 485 is configured to pull data from external data sources 465, e.g., EMR systems via the stream connector 470, e.g., a Kafka connect platform, and synchronize that data with databases 495-96. The stream processing platform 485 receives raw data from the external data sources and normalizes the data using normalizer 475, which are then harmonized via harmonizer 490. The data is then stored in patient identifiable database 495. The stream processing platform 485 also creates de-identifiable patient clinical data using de-identifier 480 which parses the data and strips all patient identifiable information. For example, the de-identifier can search data for fields associated with patient identifiable information, e.g., name, address, social security number, etc., and strip those fields of their patient information. The data is then stores in de-identified clinical database 496.

The pachyderm interface system 425 enables data scientists to search genomic database system 415 and clinical database system 420 and includes pachyderm file system (PFS) 430, pachyderm pipeline system (PPS) 435, and job workers 440. The PFS 430 is a virtual file system that also functions as a version control system for tracking changes to documents residing in local server system 410. The PFS 430 is configured to as a distributed revision control system and supports non-linear workflows and enables storage of large files sizes in object storage, e.g., within databases 450 and 495-96. The PPS 435 provides runtime management for containers and process isolation capabilities such that containerized workloads are easily parallelized across data, and scaled to utilize clustered resources. The PPS 435 further enables reading input to one database (e.g., 450) and write of output to another (e.g., databases 495-96).

FIGS. 6A-B illustrate an example data structure 600 for storing and accessing medical related information in a data store or memory according with an example embodiment of the present disclosure. The data structure 600 is structures as a biological network such as an artificial neural network such that computations are structured and stored in memory in terms of an interconnected group of artificial neurons (i.e., nodes 601). Each node is connected via edges 602. Uniquely, the data structure 600 utilizes at least three distinct types of nodes 605, 610, 615, that define a type of data that it can receive as input, store, and output. Additionally, the edges 602 include distinct edge types 620a-n such that each edge type define a type of data that it will either input to or output from a particular node 601. Additionally, the edge types 620a-n can define computing resources needed to process the data it either inputs or outputs from a node. Additionally, each of the node types 605, 610, 615 can define a physical location or logical location in memory of the data it stores such that searching and retrieval of information can occur efficiency and at an order of magnitude faster than current systems that include static nodes (i.e., where the nodes themselves do not provide an indication as to the data it holds). FIG. 6B is a graph that represents an example biological implementation of the data structure 600.

Advantageously, the data model represented by the data structure 600 enables the harmonization of data from a variety of sources each of which may use distinct semantics for similar terms. As such, each node type 601 enables annotation of tagging of data such that the data is normalized into a set of common semantics and can be harmonized by logically storing the data based on the common semantics for quick and efficient searching.

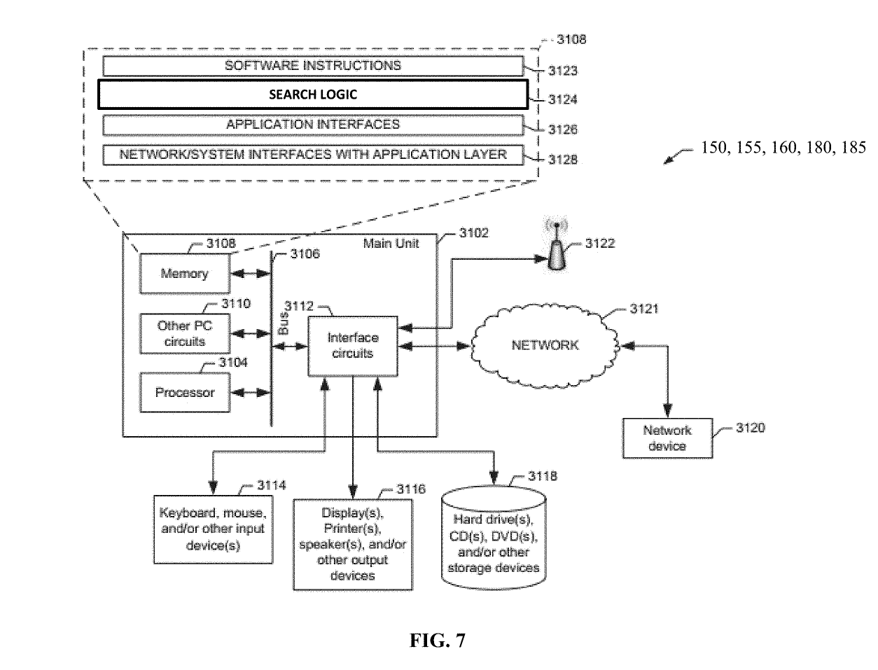

FIG. 7 illustrates a detailed block diagram of electrical systems of an example computing device (e.g., the machine learning system 150, Vocab/Ontology server(s) 155, genome database manager 160, fertility server 180, and/or user terminal 185). In this example, the computing device 150, 155, 160, 180, and/or 185 includes a main unit 3102, which preferably includes one or more processors 3104 communicatively coupled by an address/data bus 3106 to one or more memory devices 3108, other computer circuitry 3110, and one or more interface circuits 3112. The processor 3104 may be any suitable processor, such as a microprocessor from the INTEL PENTIUM® or CORE™ family of microprocessors. The memory 3108 preferably includes volatile memory and non-volatile memory. Preferably, the memory 3108 stores a software program that interacts with the other devices in the environment 100, as described above. This program may be executed by the processor 3104 in any suitable manner. In an example embodiment, memory 3108 may be part of a “cloud” such that cloud computing may be utilized by the computing device 150, 155, 160, 180, and/or 185. The memory 3108 may also store digital data indicative of documents, files, programs, webpages, patient samples, metadata, and/or medical electronic data as described above retrieved from (or loaded via) the computing device 150, 155, 160, 180, and/or 185.

Preferably, for every set of genomic data, the VCF is decomposed into separate variant data set and non-variant data set. All of the non-variant information is not stored in the genomic database, preferably only the VCF quality score and metadata associated with that non-variant given region is stored alongside the variant data. The output of a VCF format for any type of query against the genomic database is achieved by recombining the variant data, the non-variant based metadata on a reference genome dataset in real-time. The recombination of the stored variant data with the reference genome data set allows the user to query the database and achieve a report with increased speed and efficiency.

The example memory devices 3108 store software instructions 3123, search logic 3124, application interfaces 3126, user interface features, permissions, protocols, identification codes, content information, registration information, event information, and/or configurations. The memory devices 3108 also may store network or system interface features, permissions, protocols, configuration, and/or preference information 3128 for use by the computing device 150, 155, 160, 180, and/or 185. It will be appreciated that many other data fields and records may be stored in the memory device 3108 to facilitate implementation of the methods and apparatus disclosed herein. In addition, it will be appreciated that any type of suitable data structure (e.g., a flat file data structure, a relational database, a tree data structure, etc.) may be used to facilitate implementation of the methods and apparatus disclosed herein.

The interface circuit 3112 may be implemented using any suitable interface standard, such as an Ethernet interface and/or a Universal Serial Bus (USB) interface. One or more input devices 3114 may be connected to the interface circuit 3112 for entering data and commands into the main unit 3102. For example, the input device 3114 may be a keyboard, mouse, touch screen, track pad, track ball, isopoint, image sensor, character recognition, barcode scanner, microphone, and/or a speech or voice recognition system.

One or more displays, printers, speakers, and/or other output devices 3116 may also be connected to the main unit 3102 via the interface circuit 3112. The display may be a cathode ray tube (CRTs), a liquid crystal display (LCD), or any other type of display. The display generates visual displays generated during operation of the computing device 150, 155, 160, 180, and/or 185. For example, the display may provide a user interface and may display one or more webpages received from the computing device 150, 155, 160, 180, and/or 185. A user interface may include prompts for human input from a user of the computing device 150, 155, 160, 180, and/or 185 including links, buttons, tabs, checkboxes, thumbnails, text fields, drop down boxes, etc., and may provide various outputs in response to the user inputs, such as text, still images, videos, audio, and animations.

One or more storage devices 3118 may also be connected to the main unit 3102 via the interface circuit 3112. For example, a hard drive, CD drive, DVD drive, and/or other storage devices may be connected to the main unit 3102. The storage devices 3118 may store any type of data, such as the electronic data described herein, which may be used by the computing device 150, 155, 160, 180, and/or 185.

The computing device 150, 155, 160, 180, and/or 185 may also exchange data with other network devices 3120 via a connection to a network 3121 (e.g., the Internet) or a wireless transceiver 3122 connected to the network 3121. Network devices 3120 may include one or more servers, which may be used to store certain types of data, and particularly large volumes of data which may be stored in one or more data repository. A server may process or manage any kind of data including databases, programs, files, libraries, identifiers, identification codes, registration information, content information, patient samples, patient information, electronic medical data, treatment regimes, statistical data, security data, etc. A server may store and operate various applications relating to receiving, transmitting, processing, and storing the large volumes of data. It should be appreciated that various configurations of one or more servers may be used to support, maintain, or implement the computing device 150, 155, 160, 180, and/or 185 of the environment 100. For example, servers may be operated by various different entities, including operators of hospital systems, patients, drug manufacturers, service providers, etc. Also, certain data may be stored in the computing device 150, 155, 160, 180, and/or 185 which is also stored on a server, either temporarily or permanently, for example in memory 3108 or storage device 3118. The network connection may be any type of network connection, such as an Ethernet connection, digital subscriber line (DSL), telephone line, coaxial cable, wireless connection, etc.

Access the computing device 150, 155, 160, 180, and/or 185 can be controlled by appropriate security software or security measures. An individual third-party client or consumer's access can be defined by the computing device 150, 155, 160, 180, and/or 185 and limited to certain data and/or actions. Accordingly, users of the environment 100 may be required to register with the computing device 150, 155, 160, 180, and/or 185.

### EXAMPLE

Primary ovarian insufficiency (POI) is characterized by a cessation of normal ovarian function before the age of 40 and affects approximately 1% of women of reproductive age. As with menopause, POI is associated with elevated levels of follicle-stimulating hormone and deficiencies in ovarian hormones such as anti-Müllerian hormone and estrogen. These hormonal abnormalities reflect a poor ovarian reserve, and POI patients have limited fertility treatment or preservation options by the time they are diagnosed. Earlier detection of women at risk for POI or diminished ovarian reserve would increase options for family building at a younger age or fertility preservation. Closer monitoring of at-risk women would also allow for more timely intervention with hormone replacement and other therapies aimed at addressing the other health issues associated with premature decline in ovarian function.

Auto-immune disorders, endocrine abnormalities, and iatrogenic factors have been associated with POI; however, many cases are unexplained. Genetic factors are also known to contribute to POI risk, as a first-degree relative is affected by the disorder in 1-out-of-3 idiopathic cases. Despite the growing evidence-base of genetic markers associated with POI, genetic screening for these risk factors is not routine in clinical practice. In order to assess whether sufficient evidence has accumulated for the POI genetic markers reported to date to have clinical utility, a comprehensive systematic literature review and meta-analysis was conducted.

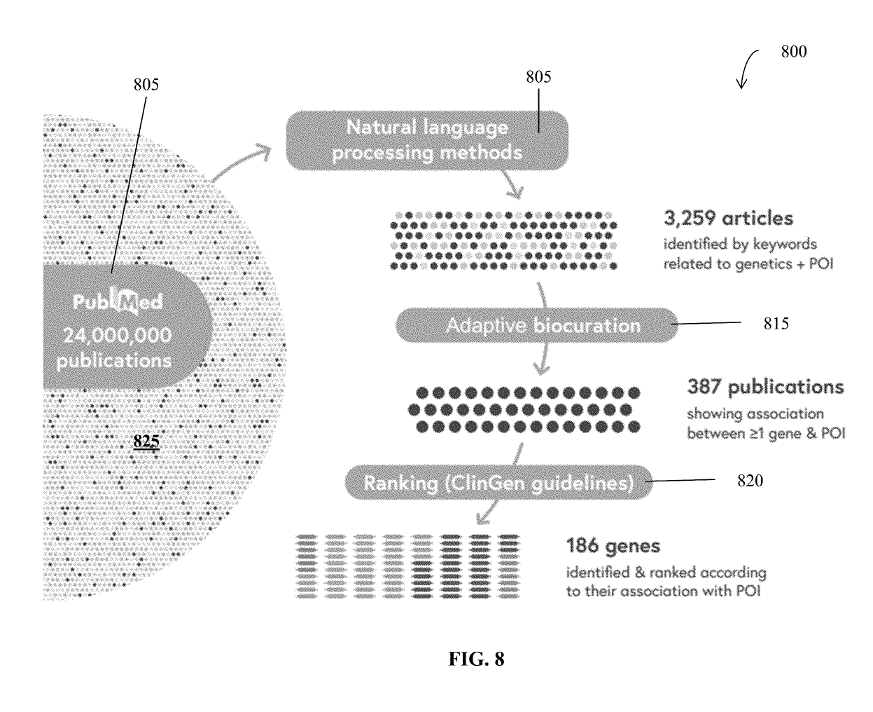

FIG. 8 is a flow diagram of a method 800 for performing a comprehensive literature review and meta-analysis using an adaptive biocuration technology (e.g., the machine learning system 150 of FIG. 1).

In the example review and analysis of FIG. 8, natural language processing algorithms are used by retrieval processors (e.g., the processors 120 of FIG. 1) to search for and identify 3,259 articles in the NCBI PubMed repository 825. The repository 825 is a publically accessible data store of medical publications that include, e.g., clinical research studies and white papers. At 805, the method 800 includes performing a search of the PubMed repository 825 using the natural language processing algorithms that receive keywords related to genetics and POI. The search retrieves articles that, at 815, are screened to remove false positives and identify false negatives using an adaptive biocuration process. In this example, the adaptive biocuration process yielded 387 “true positive” articles reporting a statistical or functional association between one or more genetic region(s) and POI. These associations are then ranked, at 820, using a classification framework (e.g., the industry-standard Clinical Genome (ClinGen) Gene-Disease Clinical Validity Classification Framework).

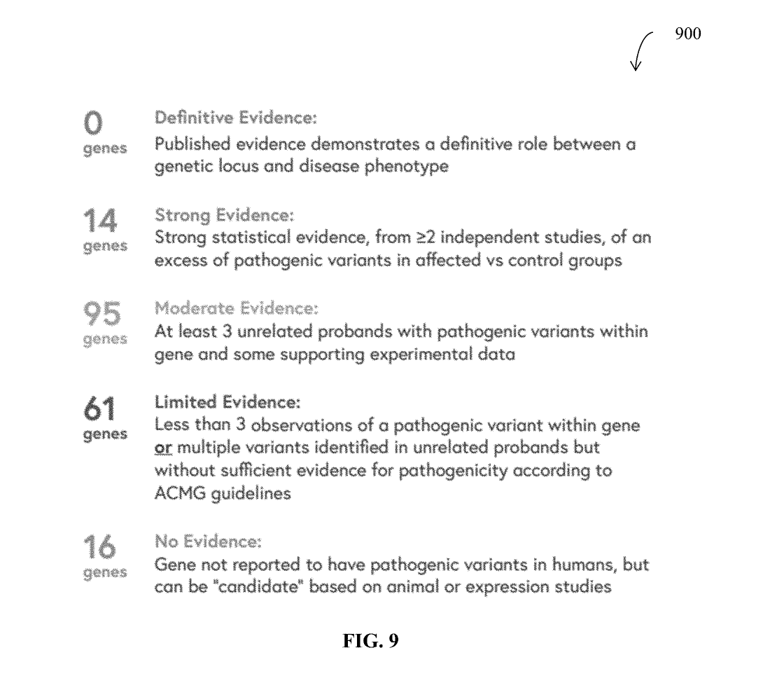

In the example study conducted using the method 800, the resulting “true positive” articles showed reports of different variants within a given gene being associated with different phenotypes (i.e. over stimulation/OHSS vs. poor stimulation/low reserve/POI). Accordingly, the insight from the reports enabled a systematic extension of the statistical validation analysis to single nucleotide variants (SNVs) within the genetic regions that adhered to the ClinGen criteria for “strong” evidence of clinical association with POI. FIG. 9 is a chart 900 that defines the levels of evidence of clinical associations with POI based on a number of gene variants of a given gene and its correlation with a strength of statistical relationship with POI based on currently available evidence.

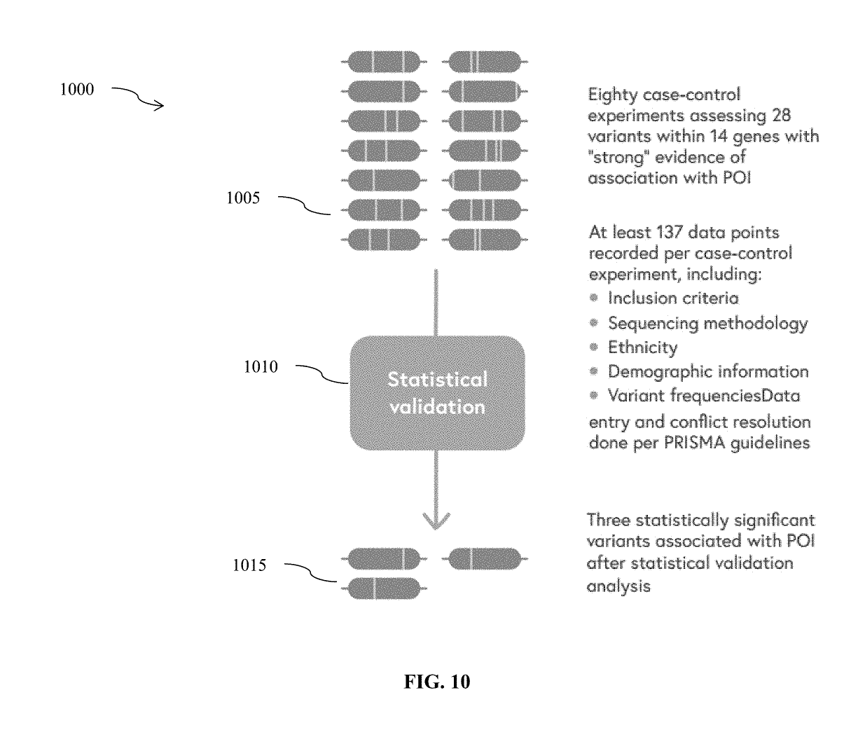

FIG. 10 illustrates flow diagram of a method 1000 for conducting a statistical validation analysis. At 1005, data points are recorded for each case-control study. In the example analysis of FIG. 10, a minimum of 137 data points were recorded for each case-control study. The method 1000, at 1010, following PRISMA guidelines, resolves any conflicts between recording data points. In the example, variants were excluded from further analysis if there were <2 published studies, overlapping cohorts, or the risk allele could not be determined based on how the information was presented in the paper. At 1015, the method 1000 determines the statistical relevance of the variants. In the example represented by FIG. 10, statistical significance was first established using a random effects model, then adjusted for multiple testing using a false discovery rate of 5%. A fertility-centric genome annotation database was used to categorize the biological functions of these genes and genetic loci.

The review and analysis revealed that no genetic regions have sufficient clinical evidence reported to meet the ClinGen guidelines for “definitive” evidence, which would require demonstrating 100% penetrance through multiple studies. Of note, genetic biomarkers classified as “definitive” are rare for most diseases and phenotypes. It was observed that fourteen genes adhered to the ClinGen guidelines for “strong” evidence of association with POI (FIG. 9). These genes have well-established roles in 1) hormone regulation, 2) immune response regulation, 3) steroidogenesis, 4) ovarian follicle development, 5) tissue remodeling, 6) glucose homeostasis, and 7) cell proliferation/differentiation. Interestingly, many of the genes that have been functionally implicated in regulating ovarian reserve did not meet the “strong” evidence criteria, in many cases because the clinical evidence is still limited. Additional studies are required to better understand whether alterations in these genes impact POI risk.

Based on ClinGen guidelines, an additional 156 genes had “moderate” or “limited” evidence of an association with POI. These genes may become elevated to stronger associations once the evidence-base is expanded. Additionally, 16 genes were categorized as “no evidence” because they had been implicated in POI risk through functional or other studies, but no human genetic studies to date have demonstrated an association with POI.

Among the 14 genetic regions with “strong” evidence of association with POI, 28 SNVs were described in a total of 80 studies and met the inclusion criteria for our meta-analyses. The statistical validation analysis demonstrated that only 3 of these variants, with risk allele frequencies <0.5 in the 1000 Genomes Project, were significantly associated with POI. Two of these variants had odds ratios (ORs)≥3.5, making them strong-effect variants according to Cohen's rule of thumb. One variant had 1.5≤OR<3.5, which according to Cohen's rule is categorized as a moderate effect.

The research illustrated by FIGS. 8-10 shows that the evidence-base for genetic markers of POI has reached the same level as many of the markers commonly used in other fields of medicine, such as oncology. These powerful markers could help identify women who are at a significantly elevated risk for being diagnosed with POI. By enabling early detection, these markers may empower women to proactively manage their reproductive health, thus maximizing their reproductive potential and mitigating the long-term consequences of delayed diagnosis and treatment.

The above-described systems and methods can be implemented in digital electronic circuitry, in computer hardware, firmware, and/or software. The implementation can be as a computer program product. The implementation can, for example, be in a machine-readable storage device, for execution by, or to control the operation of, data processing apparatus. The implementation can, for example, be a programmable processor, a computer, and/or multiple computers.

A computer program can be written in any form of programming language, including compiled and/or interpreted languages, and the computer program can be deployed in any form, including as a stand-alone program or as a subroutine, element, and/or other unit suitable for use in a computing environment. A computer program can be deployed to be executed on one computer or on multiple computers at one site.

Method steps can be performed by one or more programmable processors executing a computer program to perform functions of the disclosure by operating on input data and generating output. Method steps can also be performed by and an apparatus can be implemented as special purpose logic circuitry. The circuitry can, for example, be a FPGA (field programmable gate array) and/or an ASIC (application specific integrated circuit). Subroutines and software agents can refer to portions of the computer program, the processor, the special circuitry, software, and/or hardware that implement that functionality.

Processors suitable for the execution of a computer program include, by way of example, both general and special purpose microprocessors, and any one or more processors of any kind of digital computer. Generally, a processor receives instructions and data from a read-only memory or a random access memory or both. The essential elements of a computer are a processor for executing instructions and one or more memory devices for storing instructions and data. Generally, a computer can include, can be operatively coupled to receive data from and/or transfer data to one or more mass storage devices for storing data (e.g., magnetic, magneto-optical disks, or optical disks).

Data transmission and instructions can also occur over a communications network. Information carriers suitable for embodying computer program instructions and data include all forms of non-volatile memory, including by way of example semiconductor memory devices. The information carriers can, for example, be EPROM, EEPROM, flash memory devices, magnetic disks, internal hard disks, removable disks, magneto-optical disks, CD-ROM, and/or DVD-ROM disks. The processor and the memory can be supplemented by, and/or incorporated in special purpose logic circuitry.

To provide for interaction with a user, the above described techniques can be implemented on a computer having a display device. The display device can, for example, be a cathode ray tube (CRT) and/or a liquid crystal display (LCD) monitor. The interaction with a user can, for example, be a display of information to the user and a keyboard and a pointing device (e.g., a mouse or a trackball) by which the user can provide input to the computer (e.g., interact with a user interface element). Other kinds of devices can be used to provide for interaction with a user. Other devices can, for example, be feedback provided to the user in any form of sensory feedback (e.g., visual feedback, auditory feedback, or tactile feedback). Input from the user can, for example, be received in any form, including acoustic, speech, and/or tactile input.

The above described techniques can be implemented in a distributed computing system that includes a back-end component. The back-end component can, for example, be a data server, a middleware component, and/or an application server. The above described techniques can be implemented in a distributing computing system that includes a front-end component. The front-end component can, for example, be a client computer having a graphical user interface, a Web browser through which a user can interact with an example implementation, and/or other graphical user interfaces for a transmitting device. The components of the system can be interconnected by any form or medium of digital data communication (e.g., a communication network). Examples of communication networks include a local area network (LAN), a wide area network (WAN), the Internet, wired networks, and/or wireless networks.

The system can include clients and servers. A client and a server are generally remote from each other and typically interact through a communication network. The relationship of client and server arises by virtue of computer programs running on the respective computers and having a client-server relationship to each other.

Packet-based networks can include, for example, the Internet, a carrier internet protocol (IP) network (e.g., local area network (LAN), wide area network (WAN), campus area network (CAN), metropolitan area network (MAN), home area network (HAN)), a private IP network, an IP private branch exchange (IPBX), a wireless network (e.g., radio access network (RAN), 802.11 network, 802.16 network, general packet radio service (GPRS) network, HiperLAN), and/or other packet-based networks. Circuit-based networks can include, for example, the public switched telephone network (PSTN), a private branch exchange (PBX), a wireless network (e.g., RAN, Bluetooth, code-division multiple access (CDMA) network, time division multiple access (TDMA) network, global system for mobile communications (GSM) network), and/or other circuit-based networks.

The transmitting device can include, for example, a computer, a computer with a browser device, a telephone, an IP phone, a mobile device (e.g., cellular phone, personal digital assistant (PDA) device, laptop computer, electronic mail device), and/or other communication devices. The browser device includes, for example, a computer (e.g., desktop computer, laptop computer) with a World Wide Web browser (e.g., Microsoft® Internet Explorer® available from Microsoft Corporation, Mozilla® Firefox available from Mozilla Corporation). The mobile computing device includes, for example, a Blackberry®.

Comprise, include, and/or plural forms of each are open ended and include the listed parts and can include additional parts that are not listed. And/or is open ended and includes one or more of the listed parts and combinations of the listed parts.

One skilled in the art will realize the invention may be embodied in other specific forms without departing from the spirit or essential characteristics thereof. The foregoing embodiments are therefore to be considered in all respects illustrative rather than limiting of the invention described herein. Scope of the invention is thus indicated by the appended claims, rather than by the foregoing description, and all changes that come within the meaning and range of equivalency of the claims are therefore intended to be embraced therein.

While present disclosure has been particularly shown and described with references to example embodiments thereof, it will be understood by those skilled in the art that various changes in form and details may be made therein without departing from the scope of the present disclosure encompassed by the appended claims.

What is claimed is:

1. A machine-learning system for processing medical information, the system comprising:
a communications interface configured to access electronic medical data;
an automated retrieval processor configured to analyze the electronic medical data to identify and retrieve relevant electronic data based on predefined search criteria; and
a learning processor configured to update and optimize the automated retrieval processor based on received electronic metadata associated with the identified relevant electronic data.

2. The system of claim 1, wherein the communications interface is configured to access the electronic medical data from a public database and/or a private database.

3. The system of claim 1, wherein the communications interface is configured to access a real-time medical data feed.

4. The system of claim 1 further comprising a metadata tool configured to add electronic metadata to the identified relevant electronic data.

5. The system of claim 4 wherein the electronic metadata comprises electronic identifiers corresponding to at least one of: a false-positive marking, a false-negative marking, and/or at least one clinical data element.

6. The system of claim 5 wherein the at least one clinical data element corresponds to a predefined electronic annotation stored in a clinical data element store.

7. The system of claim 1 wherein the medical data is at least one of: an electronic structured document and/or an electronic unstructured document.

8. The system of claim 4 further comprising a phenotype/outcome data store configured to store and organize the identified relevant electronic data based on the added electronic metadata.

9. The system of claim 8 further comprising:
a genome data store configured to store and organize genomic data;
a forked loader configured to parse arbitrary file types into a predetermined format for loading genomic data into the genome data store; and
at least one set of parallelized parsers, wherein each of the at least one set of parallelized parsers is configured to parse a particular file type based on a parsing library corresponding to the particular file type.

10. The system of claim 9 further comprising a query interface tool configured to access and retrieve information from at least one of: the phenotype/outcome data store and/or the genome data store.

11. A machine-learning method for processing electronic medical information, the method comprising:
accessing electronic medical data from a public database and/or a private database;
analyzing the electronic medical data to identify and retrieve relevant electronic data based on predefined search criteria; and
performing adaptive learning based on received electronic metadata associated with the identified relevant electronic data.

12. The method of claim 11, wherein accessing electronic medical data includes accessing a real-time medical data feed.

13. The method of claim 1 wherein received electronic metadata is received from a metadata tool enabling addition of electronic metadata to the identified relevant electronic data.

14. The method of claim 13 wherein the electronic metadata comprises electronic identifiers corresponding to at least one of: a false-positive marking, a false-negative marking, and/or at least one clinical data element.

15. The method of claim 14 wherein the at least one clinical data element corresponds to a predefined electronic annotation stored in a clinical data element store.

16. The method of claim 11 wherein the medical data is at least one of: an electronic structured document and/or an electronic unstructured document.

17. The method of claim 13 further comprising storing and organizing the identified electronic relevant data based on the added electronic metadata in a phenotype/outcome data store.

18. The method of claim 17 further comprising:
storing and organize genomic data in a genome data store; and
parsing arbitrary file types into a predetermined format for loading genomic data into the genome data store.

19. The method of claim 18 wherein parsing the arbitrary file types includes performing parallel parsing using at least one set of parallelized parsers, wherein each of the at least one set of parallelized parsers is configured to parse a particular file type based on a parsing library corresponding to the particular file type.

20. The method of claim 19 further comprising enabling a query, via a query interface tool, to access and retrieve information from at least one of: the phenotype/outcome data store and/or the genome data store.
